(19) United States
(12) Patent Application Publication
(10) Pub. No.: US 2013/0023044 A1
(43) Pub. Date: Jan. 24, 2013

(54) System and Method for Fuel Generation from Algae

(71) Applicant: Cornel Gleason, Moreno Valley, CA (US)

(72) Inventor: Cornel Gleason, Moreno Valley, CA (US)

(21) Appl. No.: 13/541,265

(22) Filed: Jul. 3, 2012

Related U.S. Application Data
(60) Provisional application No. 61/509,119, filed on Jul. 19, 2011.

(51) Int. Cl. C12M 1/42

(52) U.S. Cl. 435/292.1

(57) ABSTRACT

A novel algae bioreactor and biofuel production system is presented that uses an efficient distribution and delivery mechanism for light, water, nutrient, and carbon dioxide. An efficient light delivery system uses multiple LEDs at red and blue wavelengths to enhance/optimize the algal growth. In addition, multiple light rods are immerses in large and deep bioreactor to achieve even growth of algae within the tank. A novel water/nutrient/CO2 delivery and distribution system enhances CO2 retention in the culture, produces even distribution of nutrient and results in even and rapid algae growth throughout the bioreactor tank. A nutrient delivery system is disclosed based on processing of aquatic waste for superior nutrient for algal growth. The modular design of the system allows for scaling and maintenance, for continuous operation of the plant.

### CROSS REFERENCE TO THE RELATED APPLICATIONS

This application claims the benefit of U.S. Provisional Application No. 61/509,119, filed on 19-Jul.-2011, by the same inventor and the same assignee. The U.S. Provisional Application No. 61/509,119 is hereby incorporated herein by reference in its entirety.

### BACKGROUND OF THE INVENTION

Alternative sources of energy (especially green energy) have been the subject of increased attention and focus of both commercial and governmental entities around the world. In particular, there has been a great interest in using algae to produce bio-fuel as a substitute or a complement to fossil fuel. For example see Philip T. Pienkos, “The Potential for Biofuels from Algae”, presented at the inagaural Algae Biomass Summit held Nov. 14-16, 2007 in San Francisco, Calif., http://www.nrel.gov/docs/fy08osti/42414.pdf.

Micro-algae are one of the fastest growing photosynthesizing organisms, with some strains containing large percentage of fat which can be harvested to produce biofuel through processes such as transesterification. When grown rapidly, e.g., in open ponds, microalgae store solar energy within their chemical bonds with an efficiency approaching 5% of the energy in the visible portion of the solar spectrum. This is about 10 times as great as the efficiency attainable by the major plant crops such as corn, rice, sugar cane and wheat.

However, the algae growth in open ponds suffers from the difficulty of requiring large land area to cultivate algae in shallow ponds, as the algae cells require both light and carbon dioxide (in addition to nutrient) entering from the top surface in order to grow and multiply. Therefore, the effective depth of such open ponds tends to be only about 1-2 feet. Reports (e.g., by Dr. Jian Ma in “Techno-economic analysis and engineering design consideration of algal biofuel in southern Nevada” (2011), Faculty Publications (ME), Paper 8) indicate that compared to closed photobioreactors, open ponds suffer from higher water evaporation, as well. A study by T. J. Lundquist, I. C. Woertz, N. W. T. Quinn, and J. R. Benemann, “A Realistic Technology and Engineering Assessment of Algae Biofuel production” assessed the use of waste water as the replacement for evaporative water and nutrient losses. Recently, Professor Zimmerman's team (from University of Sheffield, in UK) have used microbubbles (about 50 μm size) to float algae particles to the surface of the water and grow the algae more densely, making harvesting easier, Hanotu, J., Bandulasena, H.C. H. and Zimmerman, W. B. (2012), Microflotation performance for algal separation. Biotechnol. Bioeng., 109: 1663-1673. doi: 10.1002/bit.24449. On the other hand, tubular bioreactors for the outdoor cultivation of Nannochloropsis sp. have been shown by G. Chini Zittelli, F. Lavista, A. Bastianini, L. Rodolfi, M. Vincenzini, and M. R. Tredici, “Production of eicosapentaenoic acid by Nannochloropsis sp.cultures in outdoor tubular photobioreactors”, Journal of Biotechnology 70 (1999) 299-312.

There have been studies of algae growth in closed bioreactors with artificial lighting, e.g., using light emitting diodes (LEDs). A positive flashing light effect was observed with flashing frequencies over 1 kHz, as reported by Kyong-Hee Park and Choul-Gyun Lee in “Optimization of Algal Photobioreactors Using Flashing Lights”, Biotechnol. Bioprocess Eng. 2000, 5: 186-190. Red DDH GaAlAs LEDs from Quantum Devices Inc. (Barneveld, Wis., USA) with narrow spectral output peaks at a wavelength of approximately 680 nm were used in that study. The bioreactor design was the same as the one reported by Choul-Gyun Lee and Bernhard O. Palsson in “High-Density Algal Photobioreactors Using Light-Emitting Diodes”, Biotechnology and Bioengineering, Vol. 44, Pp. 1161-1167 (1994). That closed bioreactor had the LEDs mounted at the interior walls of a chamber radiating toward the culture at the center of the chamber, leading to a quite a small bioreactor size of about 80 cm³, and small illumination area of 100 cm² (counting both sides). At high concentrations, self-shading effect may also reduce the efficiency of algal growth, see, e.g., Susana Agusti, Carlos M. Duarte, and Jacob Kalff, “Algal cell size and the maximum density and biomass of phytoplankton”, Limnol. Oceanogr., 32(4), 1987, 983-986.

Many useful byproducts (in addition to biofuel) may be obtained by harvesting algae, e.g., see A. Robles Medina, E. Molina Grima, A. Gimenez Gimenez and M. J. Ibanez Gonzalez, “Downstream Processing of Algal Polyunsaturated Fatty Acids”, Biotechnology Advances, Vol. 16, No. 3, pp. 517-580, 1998; Owen P. Ward and Ajay Singh “Omega-3/6 fatty acids: Alternative sources of production”, Process Biochemistry 40 (2005) 3627-3652; and W. Yongmanitchai And O. P. Ward, “Growth of and Omega-3 Fatty Acid Production by Phaeodactylum tricornutum under Different Culture Conditions”, Applied And Environmental Microbiology, February 1991, p. 419-425.

The various aspects of the inventions provided in this disclosure address the challenges and shortcomings of the conventional algae growth for production of biofuels and other byproducts.

### SUMMARY OF THE INVENTION

A modular large capacity (e.g., 1000 gallon) close system bioreactor for rapid growth of algae is disclosed having light delivery system via multiple rods of LEDs surrounded by the culture in the large tank. The light wavelengths (red and blue) for LEDs are selected to match those needed for the maximum algae production, yielding efficient light delivery system. A water/nutrient/CO₂ delivery/distribution system is disclosed with enhanced retention of CO2 in the culture, even growth of algae throughout the bioreactor tank, and even distribution of nutrient via a gentle circulation within the bioreactor tank. A superior source of nutrient for algae growth is disclosed based on waste (excrement or faeces) from aquatic species (e.g., fish and shrimp) in addition to or in complement of other sources of nutrient. This reduces or eliminates dependence on petrochemicals. A CO2 capture mechanism is disclosed that reused the water contained in the exhaust for algae growth, and a system is disclosed that uses the recycle water to supplement community's water consumption resources. In addition, a system is disclosed for storage of captured CO₂ to reduce the fluctuations in CO₂ exhaust output. A modular design of the system allows scaling and isolation of the modules (in case of infection or diseases), as well as continuous operation of plant while some modules can be repaired of undergo maintenance. The modular design also allows matching the volume of resources run through the process and the logistics of the distributions of intermediaries, e.g., one centrifuge servicing multiple bioreactors running in different phases of incubation of the algae growth.

### BRIEF DESCRIPTION OF THE DRAWINGS

FIG. 1 is a schematic diagram of a biofuel generating system, in an embodiment.

FIG. 2 is a schematic of distribution system in a bioreactor, in an embodiment.

FIGS. 3(a) and 3(b) are top and cross section schematic views of the distributer, in an embodiment.

FIGS. 4(a) and 4(b) are top and cross section schematic views of the distributer, in an embodiment.

FIG. 5 is a CO₂ bubbler mechanism in an embodiment.

FIG. 6 is a schematic of biofilter in an embodiment.

FIG. 7 is a schematic of light rods using LEDs in an embodiment.

FIG. 8(a)-(e) are electrical schematic diagrams of LED strands in various embodiments.

FIG. 9 is schematic of light rods connections within bioreactor in an embodiment.

FIG. 10 is schematic of light rods supported in bioreactor in an embodiment.

FIGS. 11-13 are schematic diagrams of light rod distributions in the bioreactor used in various embodiments.

FIG. 14 is a schematic diagram of a CO₂ capture system from exhaust used in one embodiment.

FIG. 15 is a schematic diagram of an ultrasound transducer used in one embodiment. FIG. 16 is a schematic diagram of aquatic tanks used in one embodiment.

### DETAILED DESCRIPTION OF THE PREFERRED EMBODIMENTS

Note that the appendices filed with this application are the inventor's own teaching, e.g., from the earlier provisional application, and/or photos taken at inventor/assignee's own laboratory, e.g., depicting various components or setups at various times.

In order to grow algae, there are several fundamental elements: water (H₂O), nutrient, light, and carbon dioxide (CO₂):

Light+CO₂+Nutrient+Water→Algae

In turn, from Algae, other byproducts can be obtained:

Algae→Bio fuel (from fat)+Protein+Sugar

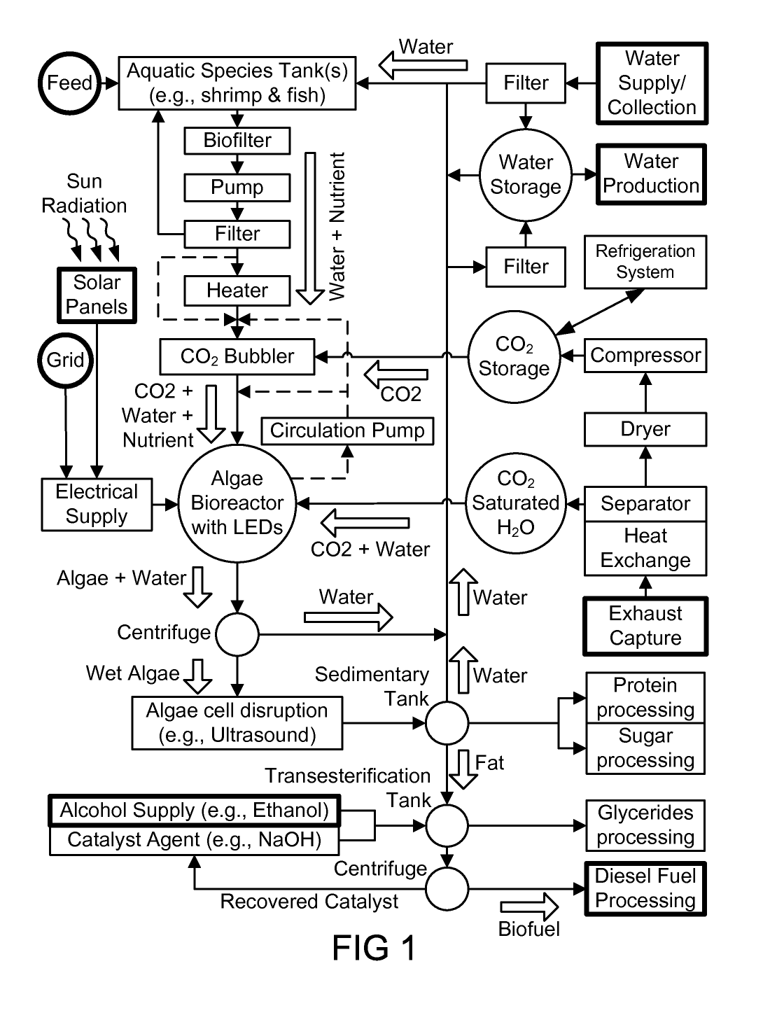

A bioreactor and system, in an embodiment of the invention, is disclosed that allows for an optimum control of light, CO₂, nutrient, and water in a modular, in an efficient and scalable design. In one embodiment, as depicted for example in FIG. 1, a system for algae growth and biofuel production uses one or more aquatic species tanks to house/grow fish or shrimp. In one embodiment, the water from these tanks runs (e.g., by a suction pump) through a biofilter which converts nitrites to nitrates via an oxidation process (e.g., by bacteria). One embodiment uses a water filter to capture large particulates (e.g., through a 12 μm paper filter). One embodiment uses an in-line heater (e.g., bypassable for example for repair purposes) to control the temperature of the water (e.g., containing nutrient) before entering the bioreactor. One embodiment uses a CO₂ bubbler to mix in CO₂ with the water flow before distribution within bioreactor. One embodiment uses artificial lighting, e.g., LEDs, within the bioreactor to control the timing/frequency/radiation power/mixture of wavelengths. In one embodiment, the power supply is backed by the grid and/or solar panels (e.g., with storage batteries), to provide the light in the wavelengths more efficient for algae growth. In one embodiment, a circulation pump is used to gently circulate and crate a flow within the bioreactor. In one embodiment, water saturated with CO₂ (e.g., from captured exhaust) is fed to bioreactor. In one embodiment, fume from an exhaust, for example from a (coal, gas, or biomass) power plant, is diverted (e.g., from stack or exhaust) by a capture mechanism. In one embodiment, the exhaust contains both CO2 and H2O, for example due to burning of methane gas:

CH₄+2O₂→CO₂+2H₂O

Depending on the water content in the exhaust, extra water may be produced as an output of the system. Given the ratio of Hydrogen atoms to Carbon atoms in biofuel is roughly about 2 to 1, potentially half of the water captured from an exhaust of a gas power plant may be produced back as output, for example, to supplement the water resources of communities. In one embodiment, a heat exchange with a cooling pond is achieved by pumping cool water from the pond to a heat exchanger to cool down the captured exhaust. In one embodiment, a separator is used to separate the liquefied water from gaseous CO₂. In one embodiment, the water collected from the separator is saturated with CO₂ (hence acidic). In one embodiment, the CO₂ saturated water is fed to bioreactor. In one embodiment, the CO₂ saturated water is mixed with water (e.g., from storage, supply, or filtered from aquatic tanks) to adjust the PH level or the concentration of CO₂. In one embodiment, the captured CO2 is dried to remove residual moisture and then compressed into one or more CO₂ high pressure tanks for later use. In one embodiment, fish and/or shrimp are harvested from aquatic tanks (as they grow) and they are processed or stored in a refrigeration system. In one embodiment, CO₂ is used as a carrier gas for the refrigeration system. CO₂ captured or supplied from other sources is used to feed into system, e.g., by a bubbler mechanism.

In one embodiment, algae water is drawn from the bioreactor and algae cells are disrupted to release fat content. In one embodiment, a centrifuge is used to extract large water content before disrupting algae cells. In one embodiment, the water removed by centrifuge is fed back to the aquatic tanks, as the residual algae particulates in the extracted water may be used as food by the aquatic species. In one embodiment, the extracted water is stored for later use of for output from system after, for example, filtration to remove algae particulates. In one embodiment, a water storage may be supplied by a water collection (e.g., from rain) or other supplies (e.g., well, streams, city water) after filtration.

In one embodiment, the disruption of algae cells are done mechanically, e.g., by press, or by ultrasound. In one embodiment, the disruption is done by adding enzymes to weaken the cell membrane. In one embodiment, after disrupting the algae cell, the fat, water, sugar and protein are separated in a sedimentary tank. In one embodiment a centrifuge is used to separate fat. In one embodiment, water is collected from the middle of the sedimentary tank, fat is collected from the top, and the sugar and protein are collected from the bottom of the tank. In one embodiment, the collected water is recycled to aquatic species tanks or stored for future use. In one embodiment, the sugar and protein are further processed to create other byproducts or for other uses (e.g., feed for animals and fermentation to ethanol).

In one embodiment, the collected fat is converted to biofuel via transesterification with an alcohol (e.g., ethanol or methanol) and a catalyst such as alkaline hydroxide (e.g., NaOH or KOH) or sodium methoxide. Transesterification is the process of exchanging the organic group R″ of an ester with the organic group R′ of an alcohol. In one embodiment, the reactions are catalyzed by the addition of an acid or base catalyst. In one embodiment, the reaction is accomplished with the help of enzymes (biocatalysts, e.g., lipases). In one embodiment, ethanol (instead of methanol) is used in this process, so that residuals would not adversely contaminate byproducts meant for human or animal consumptions. In the process of transesterification, glycerol is produced as the byproduct in addition to biofuel (i.e., Ethyl Ester of Fatty Acid):

Triglyceride+3 Ethanol→3 Ethyl Ester of Fatty Acid+Glycerol

or

R¹COO—CH₂CH(—OOCR²)CH₂—OOCR³+3CH₃CH₂OH→CH₃CH₂OOCR¹+CH₃CH₂OOCR²+CH₃CH₂OOCR³+C₃H₅(OH)₃

where R¹, R², and R³ are alkyl chains.

In one embodiment, the biofuel is collected from transesterification tank and further separated via a centrifuge. In one embodiment, the biofuel is rinsed to remove impurities and dried. In one embodiment, the catalytic agent(s) are recovered from transesterification process and reused.

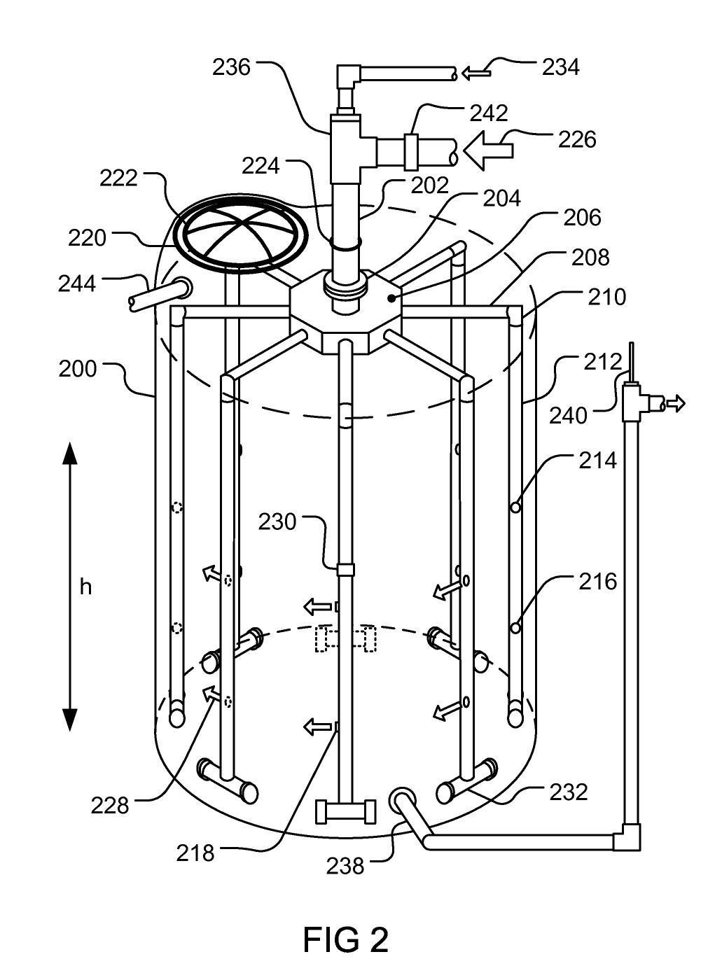

In one embodiment of this invention, as depicted for example in FIG. 2, the bioreactor chamber (or tank) (200) has a distribution mechanism for providing water, nutrient, and/or CO₂ to the algae culture. In one embodiment, the flow (226) of water, nutrient, and/or CO₂ is provided via a tube (202) from the top of the bioreactor through a sealed (224) opening. In one embodiment, this flow goes through a check valve (242). This flow is divided among multiple distributing tubes (e.g., 208) attached to a distributor (206). In one embodiment, the distributor has an octagon shape cross section for supporting 8 distributing tubes attached to the distributor. In one embodiment, the distributor is in the shape of a cylinder. In one embodiment, the distributor's cross section is triangular, square, pentagon, hexagon, heptagon, or a polygon. In one embodiment, the distributor is located inside the bioreactor. In another embodiment (not shown), the distributor is located outside the bioreactor, and the distributing tubes enter the bioreactor through sealed openings. In one embodiment, the distributor is connected to the provider tube (202) via a union flange (204) (e.g., in stainless steel), in order to allow for easier servicing, maintenance and assembly of the system, e.g., by allowing the rotation of the distribution system within the tank, accessed via an opening or manhole (220). Once, the service or assembly is concluded, the distribution assembly may be positioned and the union flange may be tightened before the normal operation. In one embodiment, the distribution tubes (e.g., 208) are turned down (e.g., via an elbow (210)) toward the bottom of the tank using one or more pieces of tubing (e.g., 212). Tube pieces are coupled (230) together in one embodiment. In one embodiment, the distributing tubes have one or more openings (e.g., 214 and 216) to let the flow enter the main chamber of the bioreactor. In one embodiment, the openings are created or placed on the distribution tubes toward the bottom of the tank or midway, so that the CO₂ introduced through the distribution assembly stays within the culture for a longer period. For example, in one embodiment, the bioreactor (200) is a 1000 gallon tank, about 6 feet high. In one embodiment, a lower opening (216) is located about 1-2 feet from the bottom of the tank; while an upper opening (214) is located about 3-4 feet from the bottom of the tank. In one embodiment, the openings are in form of a nozzle (e.g., 218). In one embodiment, the openings are holes formed or drilled into the tubes. In one embodiment, the openings (e.g., 214, 216) are positioned and distribution system is assembled so that the flow out of the holes (e.g., 228) are substantially angular (i.e., perpendicular to the radius). In one embodiment, in northern hemisphere, the orientation of the openings is set to provide the output flow in clockwise direction. In one embodiment, in southern hemisphere, the orientation of the openings is set to provide the output flow in counter-clockwise direction. In one embodiment, the openings are oriented so that the direction of the flow out of the openings coincide the general flow direction (i.e., clockwise or counter clockwise) due to the Earth's rotation on its axis (i.e., the Coriolis effect).

In one embodiment, the distribution assembly in the bioreactor is supported within the bioreactor. In one embodiment, the assembly is attached to the top or sides of the tank. In one embodiment, as for example depicted in FIG. 2, the assembly is supported by tube legs (232) at the bottom of the tank.

In one embodiment, the service access opening (manhole) (220) is covered/sealed by a door/cap (222), e.g., in order not to let CO₂ escape the tank and/or to protect the algae culture from contaminants. In one embodiment, a release value/opening (244) is provided on the tank to regulate the pressure, as well as to let liquid enter or exit (e.g., via exit 238), without impeding the flow due to over or under pressure (vacuum).

In one embodiment, bioreactor has one or more additional entries to allow input of nutrients, water, or CO₂, and other substances into the bioreactor. In one embodiment, as depicted in FIG. 2, an additional feed (234) uses the same distribution assembly, e.g., via a T connection (236). In one embodiment, CO₂ saturated water is provided via such feed (234) to the bioreactor from the CO₂ capture system.

In one embodiment, the filtered water from aquatic tanks is circulated through the bioreactor from the distribution assembly and out from an exit path (238). In one embodiment, to avoid uncontrollable siphoning of water out of the bioreactor (e.g., down to the level of aquatic tanks), a pressure reference point is made via an opening (240) to break any potential siphon.

In one embodiment, the cylindrical bioreactor tank is about 5 feet in diameter and 84″ deep. In one embodiment, the bioreactors are modularized and vertically stackable. In various embodiments, the bioreactor is installed in plant/ground/in-door, in ship, on moving/floating island.

In one embodiment, the distribution lines are connected to distributer in a star configuration, i.e., the distribution lines run out radially from distributor (as a center).

In one embodiment, the distributor comprises a ring tubing connected to the inlet tubing, and the distribution lines are connected to the distribution ring, and run vertically along the tank. In one embodiment, a distribution ring is within the tank, while in another embodiment, a distribution ring is placed outside the tank.

In one embodiment, the distributor and the main distribution lines are placed outside the bioreactor. In such a case, the flow through the distribution lines is brought into the bioreactor through sealed openings at the sides of the bioreactor, e.g., via an extension of the distribution lines or connection to installed tubing at the side of the tank. In one embodiment, the extension of a distribution line (e.g., having an opening or a nuzzle) in the bioreactor, is oriented substantially perpendicular to the radius to cause the flow enter rotationally in the culture/tank.

In one embodiment, the CO₂ inlet/distribution lines for the bioreactor are placed at the bottom of the bioreactor. In one embodiment, the inlets at the bottom of the tank are configured in a pattern (e.g., similar to those described for light rods in this disclosure).

In one embodiment as depicted for example in FIG. 2, the enclosure tank is cylindrical in shape (or has a circular cross section). In other embodiments, the enclosure tank has a polygon cross section.

In one embodiment, the sealed bioreactor tank (as for example depicted in FIG. 2) increases carbon dioxide saturation of water compared to open tanks

In one embodiment, the walls of the tank are coated white or coated by a reflective surface, so that the light generated within the bioreactor is reflected back into the tank for more efficient use of light for photosynthesis. In one embodiment, the material for various tubing include PVC, compact PVC, copper, stainless steel, aluminum, glass, plastic, or metal.

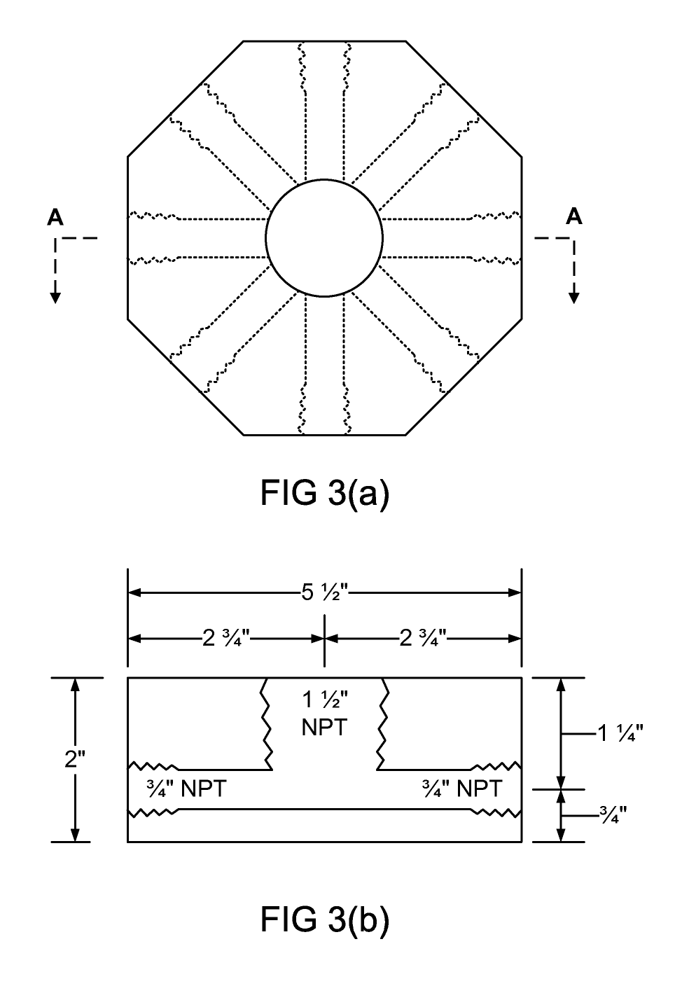

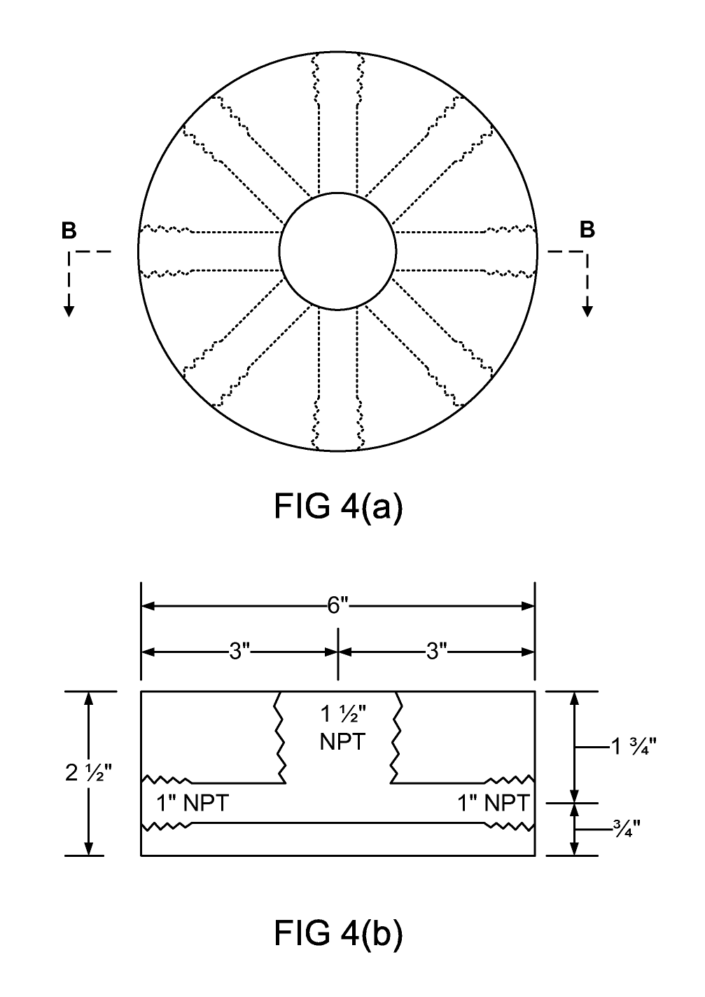

As mentioned previously, various embodiments may use various shapes for the distributor, such as octagon or circular. FIG. 3(a) depicts the top view of an octagon shape distributor with main supply from the top and 8 distribution paths on each face f octagon. Similarly, FIG. 4(a) depicts the top view of a circular (cylinder shape) distributor with main supply from the top and 8 distribution paths evenly distributed. FIGS. 3(b) and 4(b) depict cross sections and typical dimensions of a distributor for a 1000 gal tank. The main feed is for example 1¼″ diameter. The distribution lines are for example, ¾″ or 1″ diameter. In one embodiment, the distributer is threaded (for example as depicted in FIGS. 3(b) and 4(b)) for later connections with the tubes. In one embodiment, the distributor is formed by molding process. In one embodiment, tapered thread is used. In one embodiment, a standard plumbing specification, such as National Pipe Thread Tapered Thread (NPT) is used, for example, with taper rate of 1/16 per inch or ¾ inch per foot.

In one embodiment the hub or distributer is connected to the distribution lines using glue for bonding nylon.

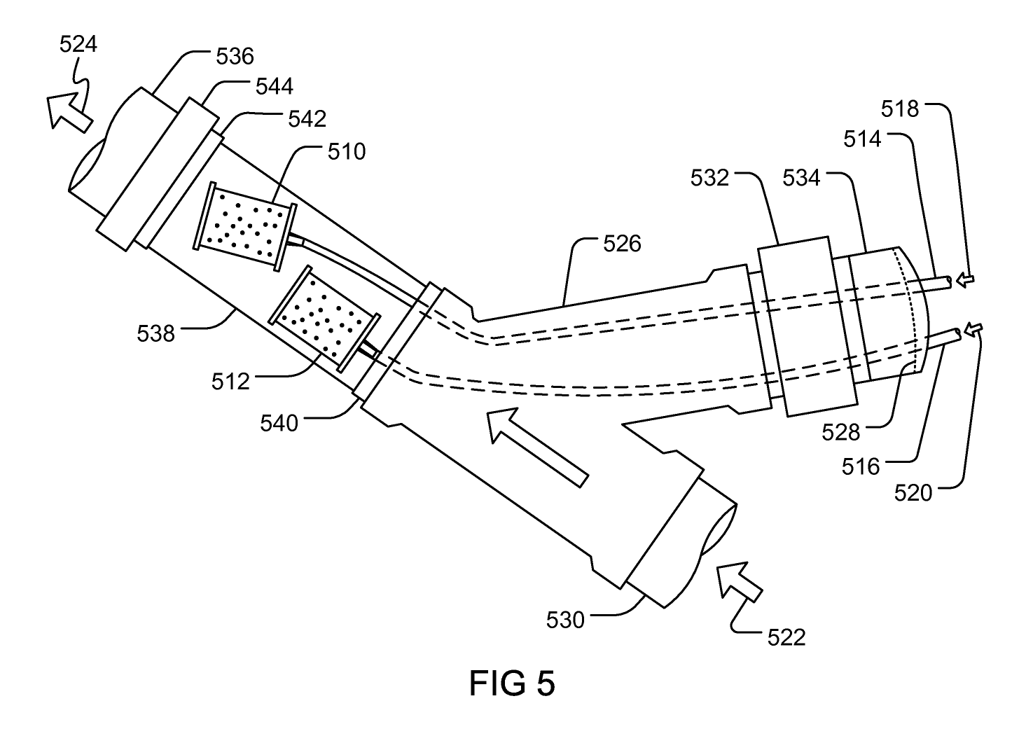

In one embodiment, as for example, depicted in FIG. 5, CO₂ gas (or other gases or mixtures) is mixed with water through a bubbler mechanism. In one embodiment, water flow is entered (522) from a tube (530) connected to a Y or T connection (526) and exits (524) from a tube (536) from the other side of Y or T. From the side connection to Y or T, the gas flow(s) (518 or 520) enter via one or more tubes (514 or 516), through a sealed (528) cap (534) which is connected to a union (532) connected to the side connection of Y or T (526). The gas tube(s) (e.g., 514 or 516) is extended pass the T or Y junction into the path of the liquid. The tubes are terminated in dispersing element (510 and 512) (e.g., air stone or a porous material) that releases the gas bubbles through holes. In one embodiment, the section of tube (538) housing the gas bubblers (510 and 512) is made of clear and transparent tube, to help observe or monitor the functionality of the bubblers. In one embodiment, the bubble is tilted, for example as depicted in FIG. 5) in order for release bubbles to move up in the direction of the flow (522 and 524).

In one embodiment, the gas bubbles produced by bubblers (e.g., ¼ to ½ inch in size) become much finer and smaller (e.g., 1 mm or less) as they travel through several feet (e.g., 20-30 feet) of tubing and dispersed in the bioreactor through the distribution assembly. The fine bubbles will have the advantage of remaining in the bioreactor tank for a longer period of times, since they tend not to reach the surface as fast as larger bubbles. In one embodiment, the bubbler includes a fine stone aerator (e.g., for 1/16″ to ⅛″ size bubbles).

In one embodiment, multiple gas bubblers (510 and 512) are placed in-line after one another (as for example depicted in FIG. 5), for example, to increase the throughput for CO₂ mixing. In one embodiment, the gas bubblers saturate water flowing through with CO₂.

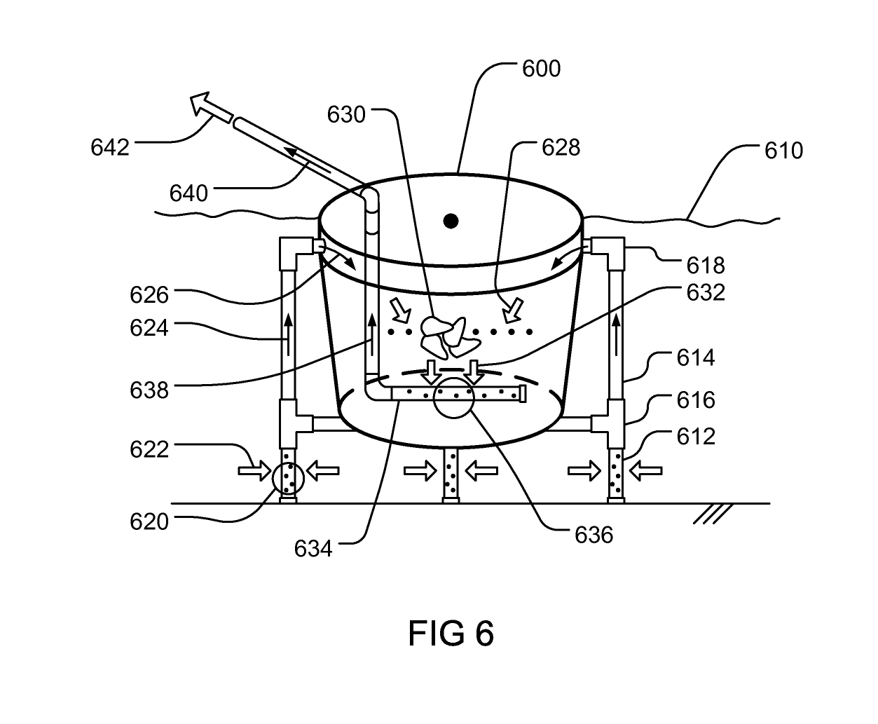

In an embodiment, as for example depicted in FIG. 6, a biofilter is used to convert aquatic waste from the aquatic tanks to form of nutrients helpful for algae growth. In one embodiment, the bio filter is immerses in an aquatic tank, in order to adapt to the tank's temperature and help flow of the water through the biofilter. In one embodiment, the biofilter (600) is supported to have its top just above the water level (610) in the aquatic tank. In one embodiment, the tubing/plumbing (e.g., 618, 614, 616, and 612) is used to help support the biofilter within the tank. In one embodiment, water from the tank flows (e.g., 622) into holes/openings (e.g., 620) in tubes (e.g., 612), e.g., via a pump (e.g., a suction pump). The flow moves through tubing (e.g., 624) into the biofilter (e.g., 626). Then, in one embodiment, the flow passes the filter before collected through holes/openings (636) on a collection tube (634), e.g., at the bottom of the biofilter. The filtered water continues to flow out (638 and 640) of the biofilter, toward for example, the suction pump or gas bubbler, on its way to the bioreactor.

In one embodiment, the biofilter is filled with lava rock. In one embodiment, the biofilter is seeded with oxidizing bacteria. In one embodiment, bacteria attach themselves to the surfaces of the lava rock. Nitrite from the aquatic waste comes into contact with bacteria in the biofilter. In one embodiment, the nitrite is converted to nitrate by the biofilter (for example, via an oxidation process). In one embodiment, the bacteria use oxygenated water to oxidize nitrites. In one embodiment, the oxygenated water is produced via bioreactor, when the during algae growth, O₂ is released in the photosynthesis process. In one embodiment, bacteria nitrosomonas or nitrobacter are used with the biofilter. Such bacteria uses energy from the oxidation of nitrite ions, NO₂⁻, into nitrate ions, NO₃⁻, to fulfill its carbon fixation requirement. In one embodiment, the biofilter is placed outside of aquatic tank.

In one embodiment, the light for photosynthesis of algae in bioreactor is provided artificially. In one embodiment, a mixture of artificial and natural light is provided for algae growth, for example, by providing light guides (for capturing sun rays) as well as artificial light (e.g., via LEDs, xenon lamp, or florescent light). In one embodiment, a mixture of LEDs emitting at wavelengths suitable for efficient algae growth is used in bioreactor. For example, for chlorophyll type A (green), the absorption peaks roughly in blue (at ˜445 nm) and red (at ˜665 nm). In one embodiment, red and blue emitting diodes are used to enhance the algae growth.

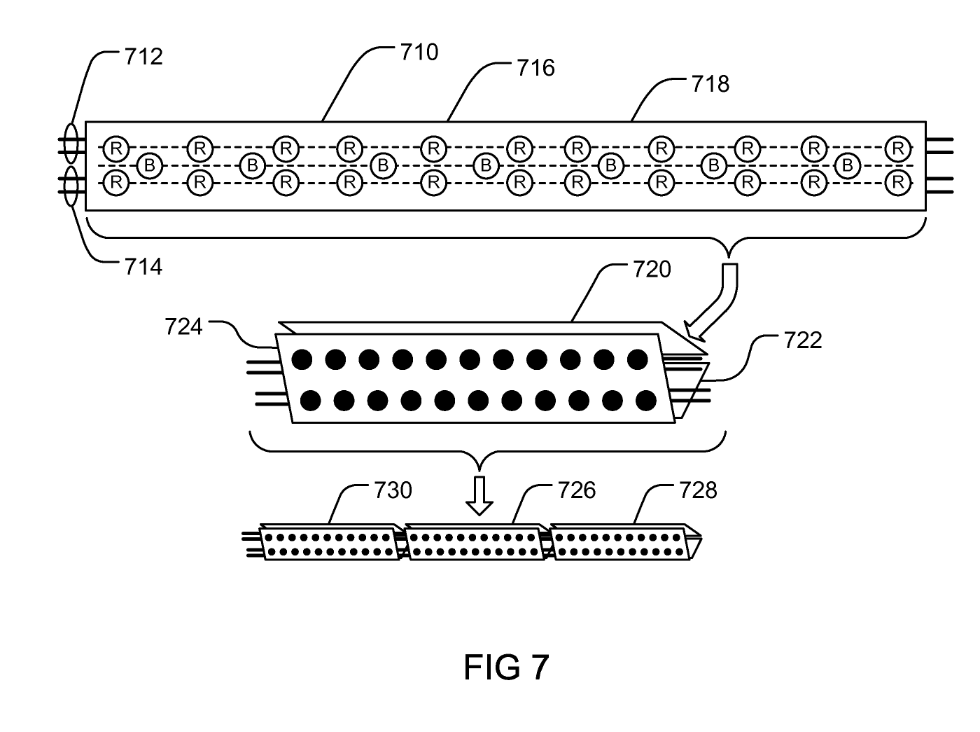

In one embodiment, as for example depicted in FIG. 7, a series of LEDs are mounted on a PCB board. In one embodiment, a large area LEDs fabricated on the substrate are used instead of single LEDs. In one embodiment, the LEDs are connected in series (e.g., see the dotted lines). For example, 11 Red LEDs (marked R in FIG. 7) in series configuration are used with power supply set about 24 volts to produce a current of about 24 mA. For example, 7 Blue LEDs (marked B in FIG. 7) is series configuration are used with power supplies set about 24 volts to produce a current of about 18 mA. In one embodiment, on the PCB board, a 100Ω resistor is used in series with each strand of 11 red LEDs, as protection against over-current. In one embodiment, a 150Ω resistor is used in series with each strand of 7 blue LEDs, as protection against over-current. In one embodiment, two strands of red LEDs are used with one strand of blue LEDs on the same PCB board. In one embodiment, the red LEDs, share a common feed supply wires, e.g., 712. In one embodiment, the blue LED feed (714) and the red LED feeds (712) are separate. In one embodiment, the null wire for both red and blue strands are used in common, so that each board carries three main wires (supply or red, supply for blue, and common null). In one embodiment, for example as depicted in FIG. 7, by 720, 722, and 724, multiple (e.g., 3, 4, 5, 6, or more) LED boards are placed (e.g., edge to edge), e.g., supported by a backing or a filler or none. In one embodiment, one or more of such sections are connected together (lengthwise) as for example shown by 730, 726, and 728, by connecting the corresponding supply and null wires, so all the red LED strands (or blue LED strands) are essentially in parallel configuration. Such a configuration forms an LED rod long enough (e.g., ˜5 feet) to provide illumination extending from about the top of water level in bioreactor to near the bottom of the tank.

In one embodiment, the LEDs are mounted in a triangle shape as shown in FIG. 7, so that the portions with wire leads are inside the triangle. In one embodiment, the effective or half angle of the light emitter is ˜120 degrees. In one embodiment, the PCB board dimension is about 21″×⅝″. In one embodiment, the supply level is set to draw about 20 mA current in each diode. In one embodiment, a light rod comprises multiple light boards (e.g., 9).

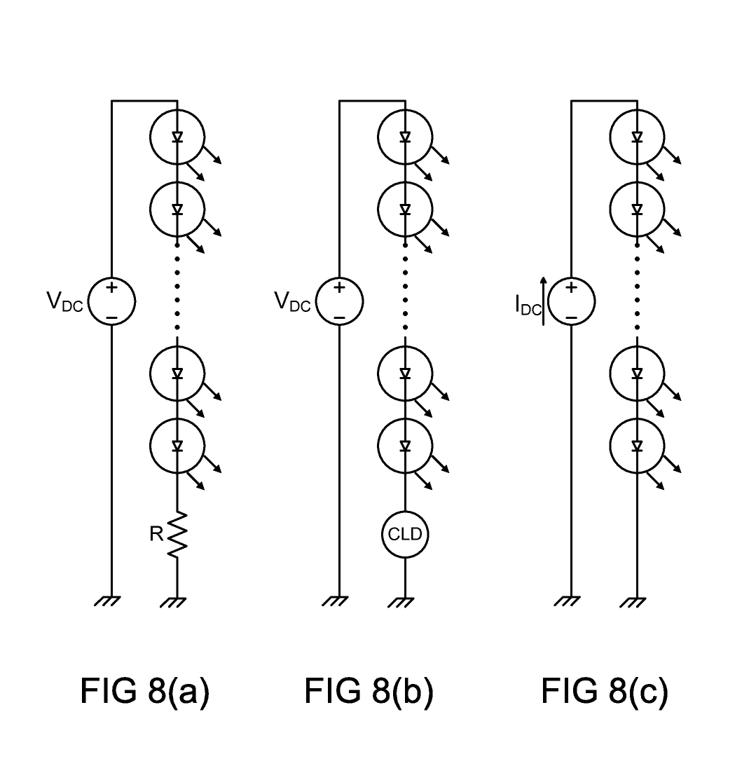

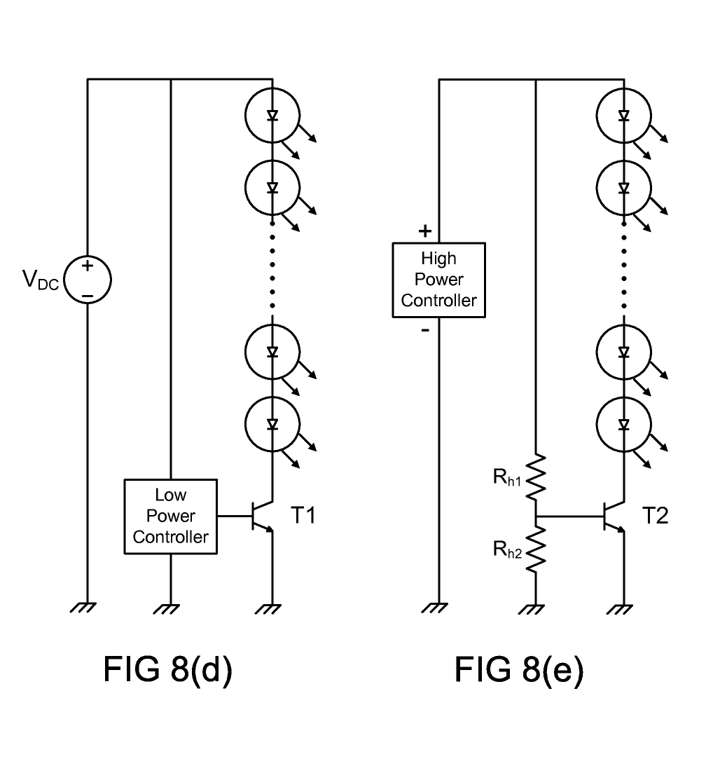

In one embodiment, as depicted for example in FIG. 8(a), the series of LEDs is protected against over-current by a resistor R (also in series). The protection is needed when used with a voltage source (V_{DC}), as the diode current to voltage has exponential characteristics. The protection may be built into a current limiter in the power supply. As depicted in FIG. 8(b), one embodiment, uses a current limiting device (or diode) (CLD), such as a JFET with its gate and drain shorted together, in order to provide a current limit for the strand. Any extra voltage would mainly appear across CLD. As depicted in FIG. 8(c), in one embodiment a current source (I_{DC}) is used as a power supply to set the current in the LED strand. In one embodiment, as depicted for example in FIG. 8(d), a transistor (T₁) (e.g., npn bipolar transistor), is used both as protection and as the controller. A low power controller component drives the base of the transistor to turn on the transistor (into saturation mode) and LEDs, or turn of the stand by applying low or zero voltage (compared to null) to the base of the transistor. In one embodiment, as depicted in FIG. 8(e), a protection is offered by a transistor (T₂) which is on (in saturation mode) when the high power controller is applying voltage to the stand. In case of over-voltage, the transistor would pick up most of the over voltage across its collector and emitter (driving it out of saturation mode).

In one embodiment, the power is supplied to the light bars in a cyclic fashion, e.g., 25% or 50% duty cycle. In one embodiment, the light bars are independently controlled to control the amount of light illumination as algae grows in the bioreactor.

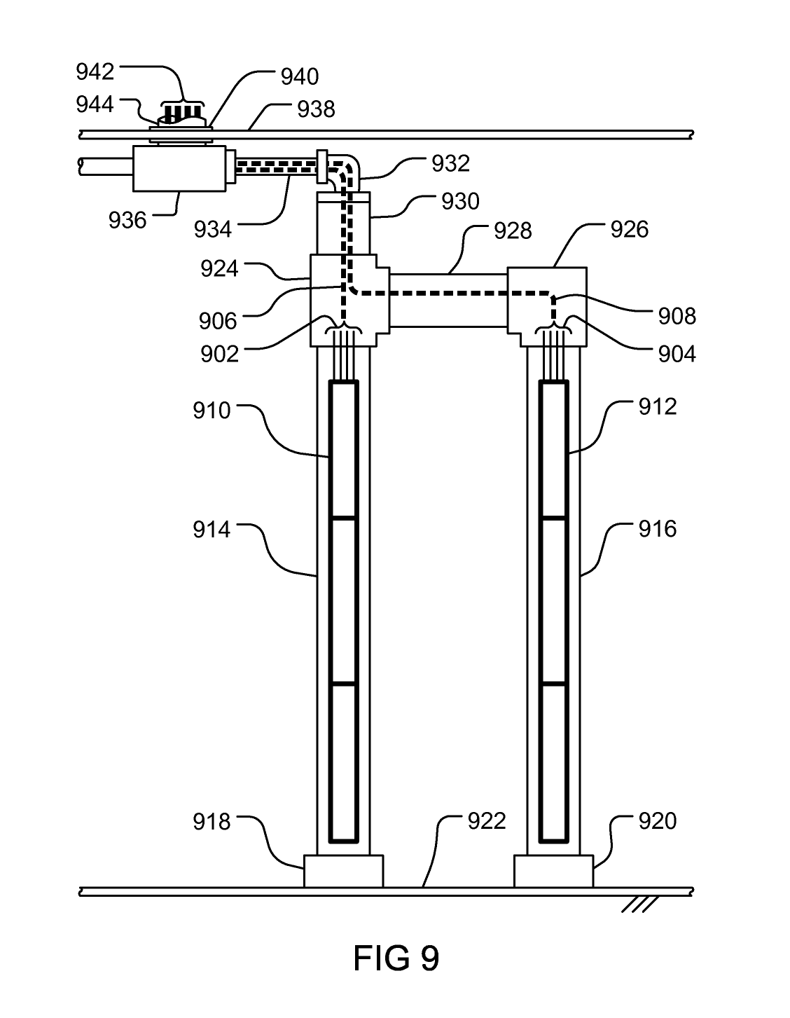

In one embodiment, as for example depicted in FIG. 9, the LED rods (e.g., 912 and 912) are placed inside protective transparent/clear tubes (e.g., 914 and 916). In one embodiment, the tubes are about 66″ designed for a 6 feet high bioreactor tank. In one embodiment, the transparent tubes are protected by ending caps (e.g., 918 and 920) to prevent the LED rod from exposure to the liquid in the tank. The insulation caps, in one embodiment, act as footing to support the light rod assembly at the bottom of the tank (922). In one embodiment, multiple light rods are supported together via tubing, as for example shown in FIG. 9. In one embodiment, the connecting tubes include elbow (926), connecting (e.g., horizontal) tube (928), and T adaptor (924), to guide the wires (e.g., 902 and 904) from the light boards (910 and 912) through (906 and 908) the connecting plumbing (930) or conduits (e.g., flexible) (934), and various adaptors (e.g., 932) to a junction box (936). In one embodiment, the junction box is sealed and placed inside the bioreactor (938), and the main supply/null wires (942) are taken from the junction box (936) to outside of the bioreactor via a conduit (944) through a sealed opening (940) in the bioreactor (938).

In one embodiment, the transparent tubes used for protecting the light bars are polycarbonate type. In one embodiment, the transparent tubes are made of glass.

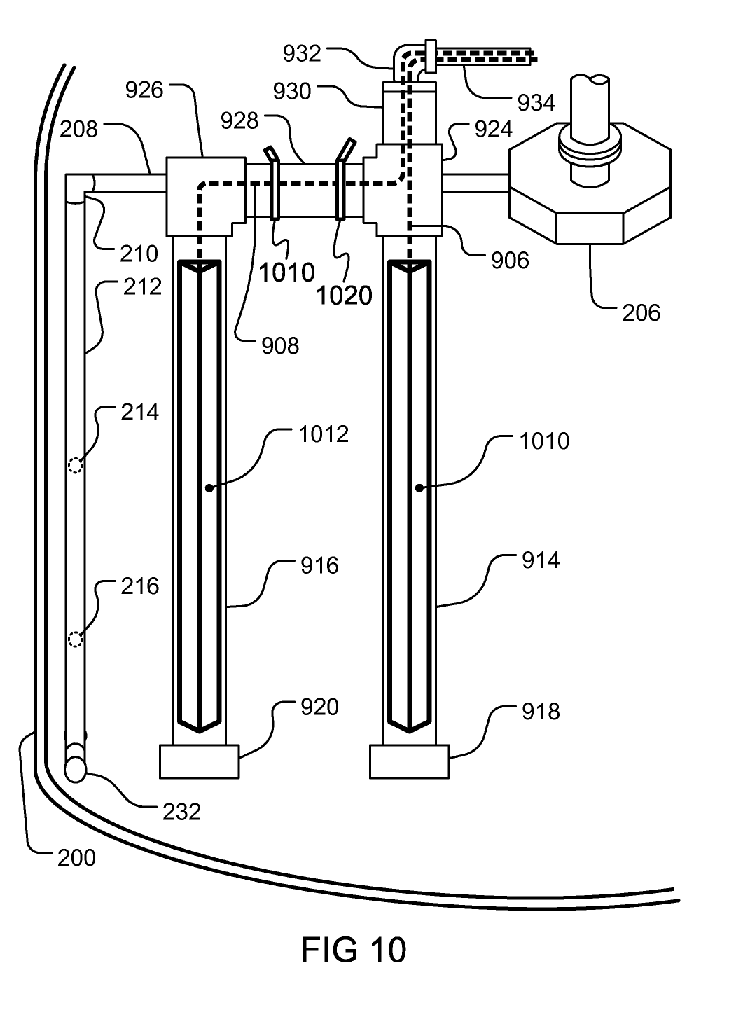

In one embodiment, as depicted for example in FIG. 10, inside the bioreactor (200), the light rod support assembly (e.g., tubing/plumbing 928, 924 and 926) which are connected to the protective clear tubes (914 and 916) containing the light bars (1010 and 1012), are fully or partially supported by the water/CO₂/nutrient distribution assembly support (e.g., 208), e.g., via fasteners or straps (1010 or 1020). In one embodiment, the light rod assembly is supported independently, e.g., by affixing or tying to the sides of the tank.

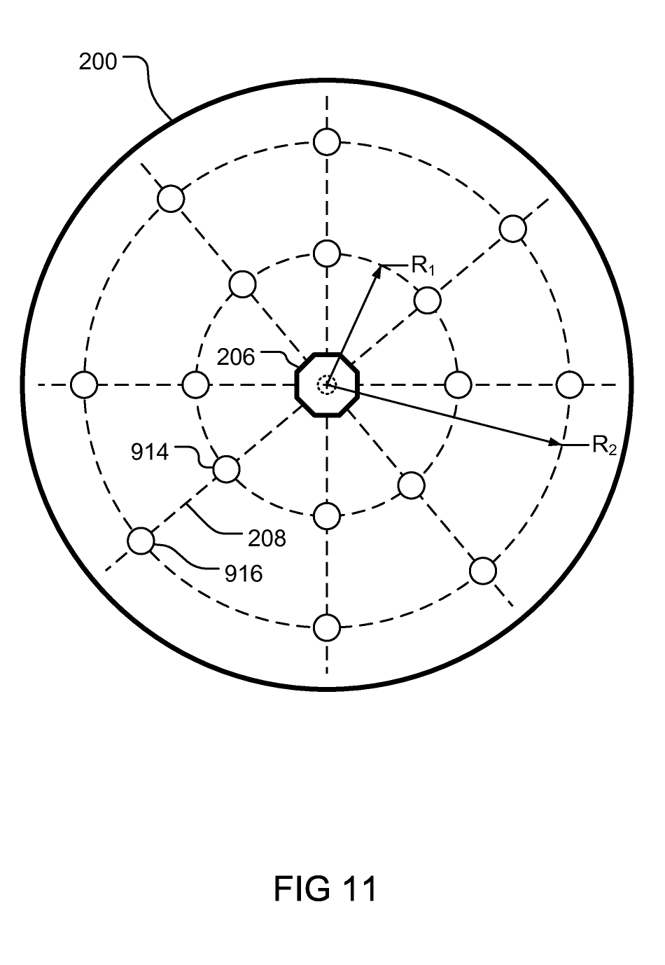

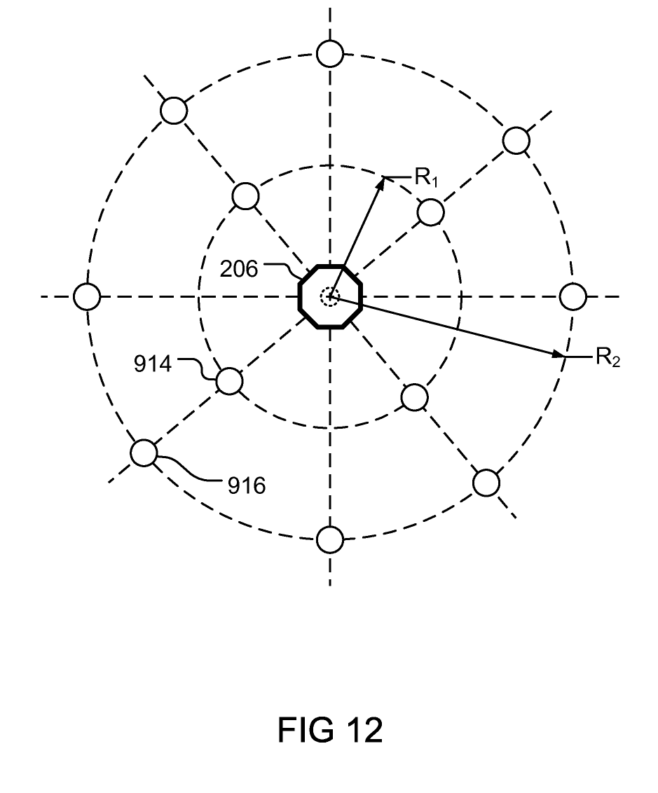

In one embodiment, as for example depicted in FIG. 11 (top view), the light rods are distributed in one, two or more radii from the center of the tank (200). In one embodiment, two pairs of light rods (e.g., 914 and 916) are supported to each water distribution line (e.g., 208) which is connected to the distributer (206), e.g., at the center. In one embodiment, for 8 distribution lines, as depicted for example in FIG. 11, there are 16 light rods in two radii (denoted as R₁ and R₂). In one embodiment, the light rods are spaced to cover (illuminate) the area around them for a close to even illumination across the cross section of the tank. In one embodiment, as depicted for example in FIG. 12, the number of light rods at each radius is not the same. For example, an inner radius has less number of light rods compared with an outer radius. In one embodiment, for example, the inner radius includes a light rod for every other light rod on an outer radius. For example for 8 light rods on an outer radius (R₂), there are 4 light rods at an inner radius (R₁).

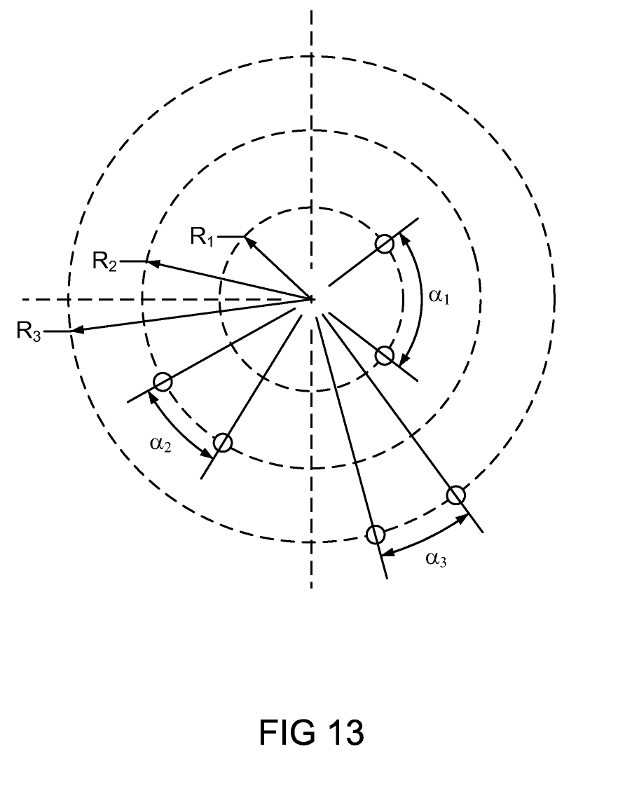

In one embodiment, as depicted for example in FIG. 13, there are multiple radii (e.g., 3 radii shown at R₁, R₂, and R₃) with various number of light rods at each radius (e.g., N₁, N₂, and N₃). In one embodiment, the light rods are angularly evenly spaced at each radius (e.g., α₁, α₂, and α₃). In one embodiment, the angular separation of neighboring light rods at each radius is about 2]0/Nᵢ, where i is the radius index. In an embodiment, the light rods are supported by holding rings from the top (or bottom).

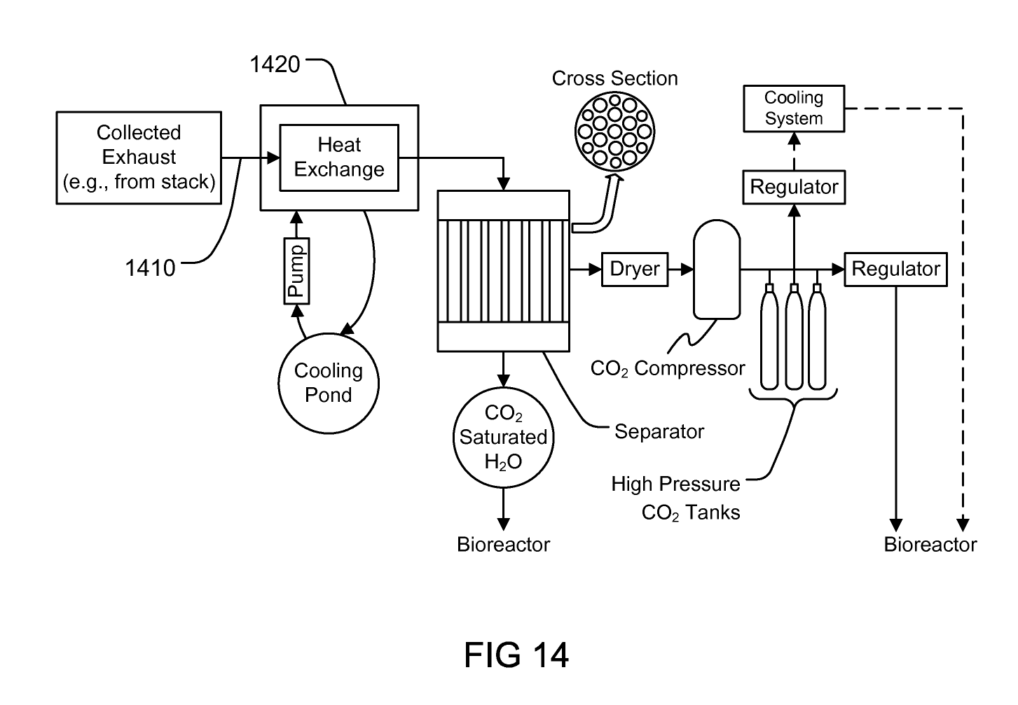

In one embodiment, as depicted for example in FIG. 14, the exhaust, e.g., from a power plant or gas (or coal) generator, is captured and brought to a heat exchanger to cool the exhaust. Exhaust (1410) typically contains CO₂ and H₂O (and some CO) in gaseous phase. In one embodiment, the heat exchange is done with a cooling pond or lake, e.g., by pumping the cool water into a water jacket (1420), and removing warm/hot water back to the cooling pond. In one embodiment, a separator (e.g., made of stainless steel) is used to separate water (condensed from vapor) from CO₂. The condensation, in one embodiment, is saturated with CO₂ (acidic), and it is directed to a storage tank to feed into bioreactor. In one embodiment, the condensation is filtered prior to feeding to the bioreactor. CO₂ (or CO) is further dried before compressing into high pressure CO₂ tanks (e.g., at 5 atm) for late and/or continuous use as feed to bioreactor (e.g., through a pressure regulator and/or bubbler). In one embodiment, CO₂ tanks are stored outside closed environment or building, with jackets surrounding the tank for safety and security.

In one embodiment, algae cells readily take in and consume CO.

In one embodiment a cooling/refrigeration system uses a limited amount/flow of CO₂ as the cooling refrigerant, for example, for storing harvested aquatic species from the aquatic tanks The refrigeration system in one embodiment, works with multiple refrigerant (e.g., Puron or Freon). In one embodiment, CO₂ from the refrigeration system is directed to bioreactor, e.g., via an in-line bubbler.

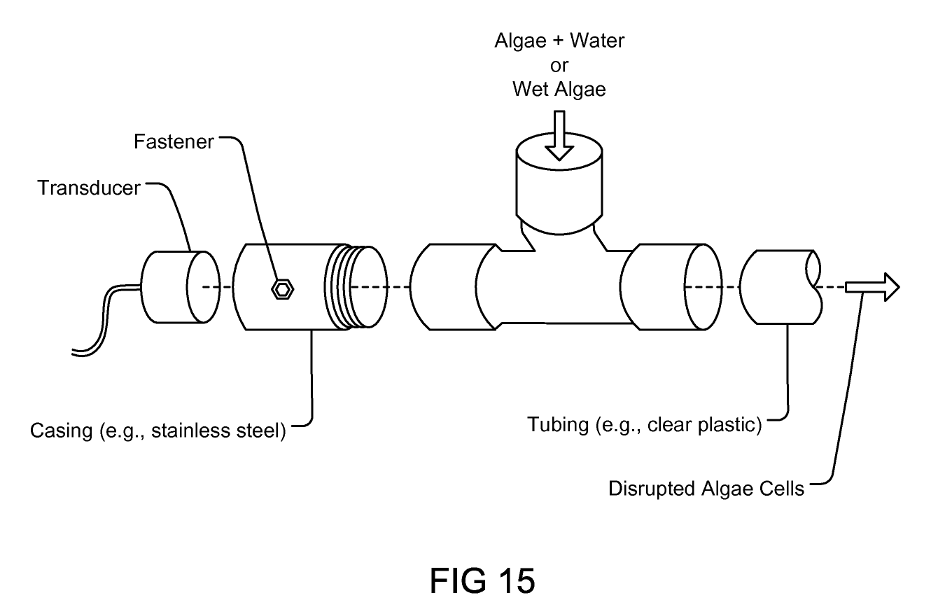

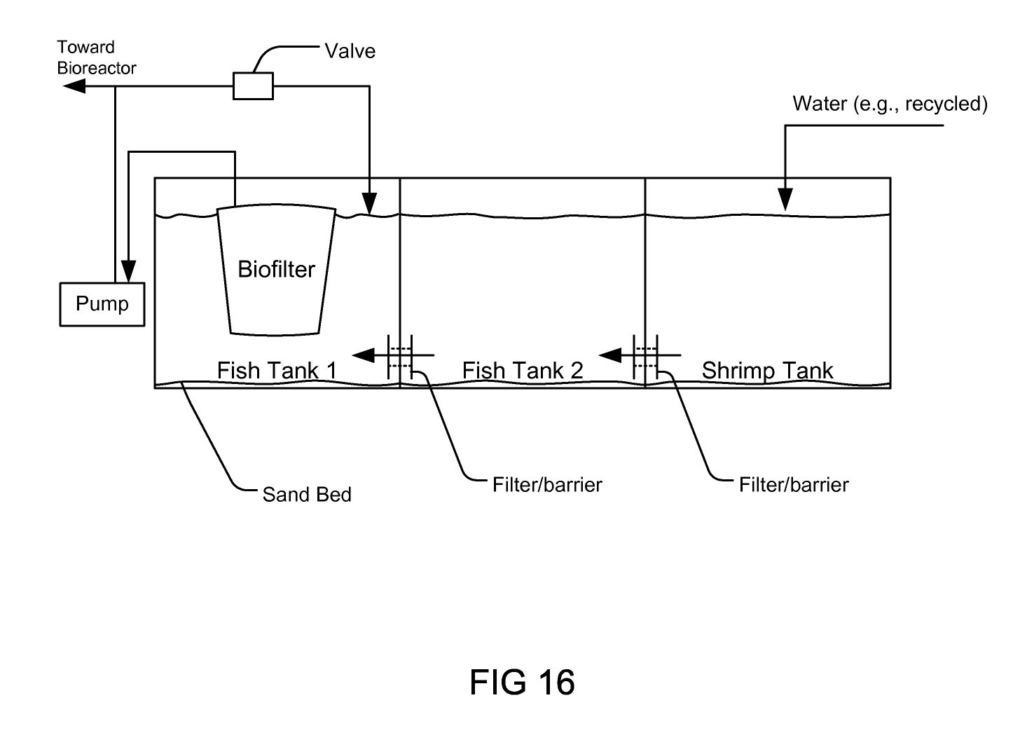

In one embodiment, as for example depicted in FIG. 15, an ultrasound transducer is used to disrupt algae cell (membrane). In one embodiment, the transducer is placed in a water tight casing (e.g., stainless steel), e.g., with acoustic sealant. In one embodiment, the transducer is supported and kept in place by one or more supports, e.g., three NYLOC fillets screws spaced at 120 degree measured from center. In one embodiment, algae water or wet algae enter from side of Y or T junction tubing connected to the casing and disrupted algae cell flow out, e.g., through a (e.g., clear) tubing (e.g., 1.5 to 2 feet) connected to Y or T. In one embodiment, an intermediate storage is used to circulate the algae water through the disruptor in multiple passes, to disrupt most of algae cells. In one embodiment, multiple ultrasound transducers or multiple frequencies are sued to disrupt the algae cells. In one embodiment, the transducer(s) produces frequencies in ranger of 1 KHz-100 KHz.

In one embodiment, the breakdown of algae cells occurs during continuous flow.

In one embodiment, as depicted in FIG. 16, multiple aquatic tanks are used in modular form to house various aquatic species. For example, in one embodiment, golden shiners are housed in one tank while shrimps are housed in another tank. Golden shiner is used in one embodiment, as it eats (e.g., algae particulates) through its last two gills and it cleans the water. In one embodiment, for example, one or more of the following species are used: red fin veiltail guppy, tropical gamboza family, fresh water fish, catfish, and/or salt water guppies.

In one embodiment, the water flows between the aquatic tanks through piping and filters/barriers (to stop aquatic species from crossing tanks) In one embodiment, the water from aquatic tank filtered through biofilter is pumped (recycled) back to aquatic tank for further pass through the biofilter. In one embodiment, the recycled water from the sedimentary tank and centrifuges are recycled back to the aquatic tank(s). In one embodiment, the top dimension of an aquatic tank is about 3 feet×8 feet. In one embodiment, the size of aquatic tank(s) is about 1,500 gallons.

In one embodiment, the separation panels in the aquatic species tank are designed to hang from the top edge of the tank. In one embodiment, a bed of sand is placed at the floor of an aquatic tank. In one embodiment, the separation panels between the tanks are set in a bed of sand.

In various embodiments, the aquatic tanks are open/close/semi-open. For example, a closed system is less susceptible to environment effects, e.g., sand storm, tornado, hurricane, air-borne diseases, or acid rain. In one embodiment, the evaporation from fish tank is controlled.

In one embodiment, plecostomus fish is used for cleaning ponds or tanks In one embodiment, gobies (e.g., Indonesian goby) are used to clear/eradicate algae off the protective tubing of light rods, e.g., if the type of algae species get in the bioreactor that sticks to the surfaces. In one embodiment, traps are set in the bioreactors to collect gobies out after the eradication.

In one embodiment, neochloris oleoabundans (a microalgae) is use to seed the bioreactor. This genius has the property that it does not tend to stick to surfaces. In one embodiment, the algae cell shape is round. In one embodiment, the algae cell size is about 5 μm.

In one embodiment, the in-line heater for feeding water/nutrient to bioreactor is set at about 75° F. In one embodiment, a 2 KW heating system, capable of 1,000 to 10,000 liter/hour flow is used.

In one embodiment, the PH is monitored to control (e.g., automatically) adjust the CO₂ feed. For example, in one embodiment, at PH level below 6, the CO₂ feed is turned off, while at PH level above 7, the CO₂ feed is turned on. In one embodiment, the CO₂ feed is partially on for PH levels between 6 and 7 based on the value of PH. In one embodiment, PH measurement is made inside the bioreactor and/or in-line with the feed to the bioreactor. In one embodiment, an automated system controls the valves and timing, using the input from various sensors (e.g., for temperature, flow meters, pressure) in the system.

In one embodiment, the PH is monitored in multiple stages via controller and sensors. If PH goes down (e.g., exceeding 1.5% weight percentage) the mixture is further diluted/mix with water, e.g., to bring the PH to 6.5 PH.

In one embodiment, the depth of water (i.e., algae water) in the bioreactor tank, for example as labeled in FIG. 2 as h, is in range of 5 to 7 feet. In one embodiment, the water pressure (from algae water) at depth of about 5 to 7 feet is still suitable for algae growth.

In one embodiment, protected cables (e.g., with multiple wires enclosed) is used within the bioreactor tank to electrically connect the light bars to a junction box or a power supply/controller. In such a case, the protective cover of the cable acts a conduit for the wires inside the cable.

In one embodiment, the lights delivery to algae culture in the bioreactor is stopped (e.g., about 4 hours in 24 hours) in normal operation. In one embodiment, cell concentration is determined/estimated during the growth process by variety of means, e.g., Secchi test, cell counts (using grids), and cell counts via optical methods.

In one embodiment, a capture storage tank (e.g., for rain water) in an open space is coated white (e.g., with painting material from Insuladd and Herculiner) to keep the water inside cool.

Note that in this disclosure, cell membrane and cell wall are meant to be used interchangeably in the context of cell disruption.

Any variations of the above teaching are also intended to be covered by this patent application.

1. A system for growth of algae in a bioreactor, said system comprising:
an enclosure tank, wherein said enclosure tank is used for holding algae culture, carbon dioxide, water, and nutrient, for algae growth in said bioreactor;
a distribution module; wherein said distribution module comprises:
a distributor,
a line feed, wherein said line feed is connected to said distributor for providing a fluid path for a fluid to said bioreactor,
wherein said fluid comprises one or more of following: water, carbon dioxide, and nutrient, and
multiple distribution lines, wherein said multiple distribution lines are connected to said distributor in a first configuration, wherein said first configuration comprises one of following: a star structure or a ring structure,
wherein said fluid path splits among said multiple distribution lines, and
wherein each distribution line of said multiple distribution lines comprises one or more openings for said fluid path to said bioreactor; and
a light delivery module, wherein said light delivery module is located within said enclosure tank;
wherein said light delivery module comprises:
multiple light rods, and
multiple conduits or protected cables, wherein each of said multiple conduits or protected cables is used to provide a portion of path for electrical connection between one or more of said multiple light rods and a power source,
wherein said multiple light bars emit radiation used in photosynthesis by algae within said bioreactor,
wherein said multiple light bars are immersed within said enclosure tank and distributed in a second configuration, and
wherein said second configuration provides an illumination coverage across a cross section of inside of said enclosure tank for algae growth in said bioreactor.

2. The system for growth of algae in a bioreactor as recited in claim 1, wherein said enclosure tank has a circular cross section.

3. The system for growth of algae in a bioreactor as recited in claim 1, wherein said enclosure tank is vertically stackable.

4. The system for growth of algae in a bioreactor as recited in claim 1, wherein said distributor has a polygon or circular cross section.

5. The system for growth of algae in a bioreactor as recited in claim 1, wherein said multiple distribution lines are evenly spaced with respect to said distributor.

6. The system for growth of algae in a bioreactor as recited in claim 1, wherein said distributor is located inside said enclosure tank.

7. The system for growth of algae in a bioreactor as recited in claim 1, wherein said distributor and said multiple distribution lines are placed outside said enclosure tank.

8. The system for growth of algae in a bioreactor as recited in claim 1, wherein one of said one or more openings comprises a nuzzle.

9. The system for growth of algae in a bioreactor as recited in claim 1, wherein one of said one or more openings is formed as a hole in said each distribution line.

10. The system for growth of algae in a bioreactor as recited in claim 1, wherein said one or more openings are oriented to pass flow perpendicular to radial direction with respect to said enclosure tank.

11. The system for growth of algae in a bioreactor as recited in claim 1, said system further comprising an exit path for algae water from said enclosure tank to a centrifuge or an algae cell disrupter.

12. The system for growth of algae in a bioreactor as recited in claim 1, wherein each of said multiple light rods is protected via a transparent cover.

13. The system for growth of algae in a bioreactor as recited in claim 12, wherein said transparent cover is sealed to prevent fluid from said bioreactor reaching any of said multiple light rods.

14. The system for growth of algae in a bioreactor as recited in claim 1, wherein said multiple light rods comprise light emitting diodes.

15. The system for growth of algae in a bioreactor as recited in claim 14, wherein said light emitting diodes comprise blue and red diodes.

16. The system for growth of algae in a bioreactor as recited in claim 14, wherein said light emitting diodes comprise blue diodes.

17. The system for growth of algae in a bioreactor as recited in claim 1, said system further comprising a control module for controlling the timing, duty cycle, and light illumination for said multiple light rods.

18. The system for growth of algae in a bioreactor as recited in claim 1, said system further comprising a bubbler to mix carbon dioxide gas with water for feeding to said bioreactor.

19. The system for growth of algae in a bioreactor as recited in claim 1, said system further comprising an aquatic tank for producing aquatic waste as a source of nutrient for algae growth.

20. The system for growth of algae in a bioreactor as recited in claim 1, said system further comprising a carbon dioxide capture system for providing water saturated with carbon dioxide as a feed to said bioreactor.
